(19) United States
(12) Patent Application Publication
(10) Pub. No.: US 2023/0167408 A1
(43) Pub. Date: Jun. 1, 2023

(54) METHODS AND COMPOSITIONS FOR MAINTAINING AND EXPANDING HEMATOPOIETIC STEM CELLS

(71) Applicant: Trailhead Biosystems Inc., Cleveland, OH (US)

(72) Inventor: Angelica M. GOMES UELTSCHY, Shaker Heights, OH (US)

(21) Appl. No.: 18/071,388

(22) Filed: Nov. 29, 2022

Related U.S. Application Data
(60) Provisional application No. 63/284,360, filed on Nov. 30, 2021.

(51) Int. Cl. C12N 5/0789

(52) U.S. Cl. CPC C12N 5/0647; C12N 2501/125; C12N 2501/145; C12N 2501/15; C12N 2500/38; C12N 2500/42; C12N 2501/42

(57) ABSTRACT

Methods for maintaining and expanding human CD34+ hematopoietic stem cells (HSCs) are provided using chemically-defined culture media that allow for expansion of HSCs in as little as six days. Culture media, isolated cell populations and kits are also provided.

### RELATED APPLICATIONS

This application claims priority to U.S. Provisional Application No. 63/284,360, filed Nov. 30, 2021. The entire contents of which is hereby incorporated by reference.

### BACKGROUND OF THE INVENTION

Hematopoietic stem cells (HSCs) are pluripotent, self-renewing cells that give rise to the entire hematopoietic system, including cells of the myeloid and lymphoid lineages. HSCs are rare cells naturally found in bone marrow and umbilical cord blood, and even more rarely in peripheral blood. HSCs are typically defined by the expression, or lack of expression, of particular markers, including expression of CD34 and lack of expression of Lineage-specific markers and CD38 (Lin-CD34+CD38−). Allogeneic HSCs are used in stem cell transplantation as a means of treatment for serious hematological diseases, including cancer, such as leukemias and lymphomas, and autoimmune disorders. The ability to use HSCs in transplantation, however, requires the ability to isolate, expand and maintain HSCs in culture, given their very limited occurrence naturally.

Various culture systems for expanding CD34+ HSCs have been described, typically including a culture medium supplemented with stem cell factor (SCF) and thrombopoietin (TPO), along with other cytokines and small molecules. For example, Nishino et al. report expanding HSCs by culturing for seven days in a medium containing SCF, TPO and garcinol, a potent inhibitor of histone acetyltransferase (Nishino et al. (2011) PLoS One 6:e24298). Himburg et al. report expanding HSCs by culturing in media containing SCF, TPO, Flt-3 ligand and pleiotrophin, the latter of which activates PI3K signaling (Himburg et al. (2010) Nat. Med. 16:475-482). CD34+ HSCs from umbilical cord blood have been expanded ex vivo by culture in a base media containing SCF, TPO, Flt-3 ligand and low density lipoproteins to which was added an inhibitor of the JNK pathway (Xiao et al. (2019) Cell. Discovery 5:2). Alternatively, CD34+ HSCs from umbilical cord blood were expanded ex vivo by culture in a media containing SCF, TPO, Flt-3 and IL-3 for 16 hours followed by further culture for seven days with the deacetylase inhibitor valproic acid (VPA) (Papa et al. (2019) J. Vis. Exp. DOI:10.3791/59532).

Aryl hydrocarbon receptor antagonists, such as the purine derivative SR1, have been reported to promote the expansion of HSCs (Boitano et al. (2010) Science 329:1345-1348).

Antagonism of peroxisome proliferator-activated receptor (PPAR)-γ has been reported to promote the expansion of HSCs (Guo et al. (2018) Nat. Med. 24:360-367).

More recently, addition of an inhibitor of the NFκB signaling pathway for 10 days has been reported to increase expansion of HSCs in culture (Sun et al. (2021) Exp. Cell. Res. 399:112468).

Furthermore, through screening of a panel of 186 chemicals, the combination of CHIR-99021 (GSK3 inhibitor), Forskolin (cAMP activator) and OAC1 (Oct4 activator) was identified as a chemical cocktail that supported the maintenance of HSCs in culture (Jiang et al. (2018) Cell. Discovery 4:59).

Accordingly, while some progress has been, there remains a need for efficient and robust methods and compositions for expanding and maintaining HSCs in culture.

### SUMMARY OF THE INVENTION

This disclosure provides methods of maintaining and expanding human hematopoietic stem cells (HSCs), e.g., from cord blood or bone marrow cells, using chemically-defined culture media that allows for expansion of CD34+ HSCs in as little as six days of culture. The culture media lacks serum and comprises small molecule agents that either agonize or antagonize particular signaling pathway in stem cells such that expansion and self-renewal along the CD34+ HSC lineage is promoted, leading to expression of HSC-associated biomarkers. The methods of the disclosure have the advantage that they significantly shorten the time needed to expand CD34+ HSC. Moreover, the use of small molecule agents in the culture media allows for precise control of the culture components.

Accordingly, in one aspect, the disclosure pertains to a method of maintaining or expanding human CD34+ hematopoietic stem cells (HSCs), the method comprising culturing human CD34+ HSCs in a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor.

In an embodiment, the c-kit ligand is stem cell factor (SCF).

In an embodiment, the TPOR agonist is thrombopoietin (TPO). In other embodiments, the TPOR agonist is eltrombopag, TA-316, TPO agonist 1, avatrombopag or lusutrombopag.

In an embodiment, the TGFβ pathway agonist is Activin A. In another embodiment, the TGFβ pathway agonist is alantolactone.

In an embodiment, the antioxidant is a form of vitamin C (ascorbic acid), such as L-ascorbic acid phosphate sesquimagnesium salt hydrate, a stable form of vitamin C (Vit. C). In other embodiments, the antioxidant is ascorbic acid, glutathione, ebeselen, N-acetyl-L-cysteine or α-tocopherol.

In an embodiment, the bioactive phospholipid is lysophosphatidic acid (LPA). In other embodiments, the bioactive phospholipid is sphingosine-1-phosphage (SIP), ceramide-1-phosphate (C1P) or lysophosphatidylcholine (LPC).

In an embodiment, the AhR agonist is 6-Formylindolo[3,2-b]carbazole (FICZ). In other embodiments, the AhR agonist is Norisoboldine, Pifithrin-α hydrobromide, MeBIO, ITE or 10-C1-BBQ.

In an embodiment, the Notch agonist is Yhhu 3792. In other embodiments, the Notch agonist is Jagged 1-2 or DLL1-4.

In an embodiment, the HDAC inhibitor is valproic acid (VPA). In other embodiments, the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1.

In an embodiment, the method of maintaining or expanding human CD34+ hematopoietic stem cells (HSCs) comprises culturing human CD34+ HSCs in a culture media comprising Stem Cell Factor (SCF), thrombopoietin (TPO), Activin A, Vitamin C, lysophosphatidic acid (LPA), 6-Formylindolo[3,2-b]carbazole (FICZ), Yhhu 3792 and valproic acid (VPA). In an embodiment, SCF is present at a concentration of 10 ng/ml, TPO is present at a concentration of 100 ng/ml, Activin A is present at a concentration of 20 ng/ml, Vitamin C is present at a concentration of 100 uM, LPA is present at a concentration of 200 nM, FICZ is present at a concentration of 500 nM, Yhhu 3792 is present at a concentration of 750 nM and VPA is present at a concentration of 150 uM.

In the methods and compositions of the disclosure, the CD34+ HSCs can be from, for example, umbilical cord blood or bone marrow.

In an embodiment, the CD34+ HSCs are cultured in the media described herein for at least six days (e.g., for 7 days, or 1 week, or longer).

In an embodiment, the CD34+ HSCs have a phenotype of Lin-CD34+CD38−CD45RA−CD90+.

In another aspect, the disclosure pertains to a culture media for expanding or maintaining human CD34+ hematopoietic stem cells (HSCs) comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor.

In an embodiment, the c-kit ligand is stem cell factor (SCF).

In an embodiment, the TPOR agonist is thrombopoietin (TPO). In other embodiments, the TPOR agonist is eltrombopag, TA-316, TPO agonist 1, avatrombopag or lusutrombopag.

In an embodiment, the TGFβ pathway agonist is Activin A. In another embodiment, the TGFβ pathway agonist is alantolactone.

In an embodiment, the antioxidant is vitamin C. In other embodiments, the antioxidant is ascorbic acid, glutathione, ebeselen, N-acetyl-L-cysteine or α-tocopherol.

In an embodiment, the bioactive phospholipid is lysophosphatidic acid (LPA). In other embodiments, the bioactive phospholipid is sphingosine-1-phosphage (SIP), ceramide-1-phosphate (C1P) or lysophosphatidylcholine (LPC).

In an embodiment, the AhR agonist is 6-Formylindolo[3,2-b]carbazole (FICZ). In other embodiments, the AhR agonist is Norisoboldine, Pifithrin-α hydrobromide, MeBIO, ITE or 10-C1-BBQ.

In an embodiment, the Notch agonist is Yhhu 3792. In other embodiments, the Notch agonist is Jagged 1-2 or DLL1-4.

In an embodiment, the HDAC inhibitor is valproic acid (VPA). In other embodiments, the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1.

In an embodiment, the culture media comprises Stem Cell Factor (SCF), thrombopoietin (TPO), Activin A, Vitamin C, lysophosphatidic acid (LPA), 6-Formylindolo[3,2-b]carbazole (FICZ), Yhhu 3792 and valproic acid (VPA). In an embodiment, SCF is present at a concentration of 10 ng/ml, TPO is present at a concentration of 100 ng/ml, Activin A is present at a concentration of 20 ng/ml, Vitamin C is present at a concentration of 100 uM, LPA is present at a concentration of 200 nM, FICZ is present at a concentration of 500 nM, Yhhu 3792 is present at a concentration of 750 nM and VPA is present at a concentration of 150 uM.

In yet another aspect, the disclosure pertains to an isolated cell culture of human CD34+ hematopoietic stem cells (HSCs), the culture comprising: human CD34+ HSCs cultured in a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor. Suitable agents include those described above.

In yet another aspect, the disclosure pertains to a method of maintaining human CD34+ long term hematopoietic stem cells (LT-HSCs) comprising culturing human CD34+ HSCs in a culture media comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO). Non-limiting examples of suitable agents, and concentrations therefor, include those described above. In an embodiment, the TGFβ pathway agonist is Activin A, the bioactive phospholipid is lysophosphatidic acid (LPA), the AhR agonist is 6-Formylindolo[3,2-b]carbazole (FICZ)and the HDAC inhibitor is valproic acid (VPA). In an embodiment, the LT-HSCs are CD34+ cells that also express CRHBP, HOPX and LMO2.

In another aspect, the disclosure provides a culture media for maintaining human CD34+ long term hematopoietic stem cells (LT-HSCs) comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO). In another aspect, the disclosure provides an isolated cell culture of human CD34+ long term hematopoietic stem cells (LT-HSCs), the culture comprising: human CD34+LT-HSCs cultured in a culture media comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO).

Other features and advantages of the invention will be apparent from the following detailed description and claims.

### BRIEF DESCRIPTION OF THE DRAWINGS

FIG. 1 shows results from an HD-DoE model of an 8-factor experiment optimized for maximum expression of GATA2. The upper section of the model shows the prediction of expression level of pre-selected 53 genes when optimized for GATA2. The lower section of the model shows the effectors that were tested in this model and their contribution to maximum expression of GATA2. The value column refers to required concentration of each effector to mimic the model.

FIG. 2 shows the dynamic profile of expression levels of GATA2, CRHBP and MEG3 genes relative to the concentration of 8 effectors tested. The positive impact of LPA, FCIZ, Vit. C and Activin A on expression of GATA2 and their factor contribution is shown by the slope of the plots for each effector.

FIG. 3 shows results from an HD-DoE model of an 8-factor experiment optimized for maximum expression of CRHBP. The upper section of the model shows the prediction of expression level of pre-selected 53 genes when optimized for CRHBP. The lower section of the model shows the effectors that were tested in this model and their contribution to maximum expression of CRHBP. The value column refers to required concentration of each effector to mimic the model.

FIG. 4 shows the dynamic profile of expression levels of CRHBP, GATA2 and MEG3 genes relative to the concentration of 5 effectors tested. The positive impact of VPA on expression of all three genes and their factor contribution is shown by the slope of the plots for each effector.

FIG. 5 shows results from an HD-DoE model of an 8-factor experiment optimized for maximum expression of CRHBP. The upper section of the model shows the prediction of expression level of pre-selected 53 genes when optimized for CRHBP. The lower section of the model shows the effectors that were tested in this model and their contribution to maximum expression of CRHBP. The value column refers to required concentration of each effector to mimic the model.

FIGS. 6A-B show the dynamic profile of expression levels of GATA2, HOXA5 and MEG3 genes relative to the concentration of 4 validated effectors for HSC expansion (FICZ, LPA, Vit. C, Activin A). FIG. 6A shows expression levels of genes of interest in the presence of all five finalized effectors. FIG. 6B shows expression levels of genes of interest in the absence of one of the finalized effectors at a time while others are present.

FIGS. 7A-B show the dynamic profile of expression levels of GATA2, HOXA5 and MEG3 genes relative to the concentration of 1 validated effector for HSC expansion (VPA). FIG. 7A shows expression levels of genes of interest in the presence of VPA. FIG. 7B shows expression levels of genes of interest in the absence of VPA.

FIGS. 8A-B show the dynamic profile of expression levels of GATA2, HOXA5 and MEG3 genes relative to the concentration of 1 validated effector for HSC expansion (Yhhu 3792). FIG. 8A shows expression levels of genes of interest in the presence of Yhhu 3792.

FIG. 8B shows expression levels of genes of interest in the absence of Yhhu 3792.

FIGS. 9A-B show the results of flow cytometry analyses of cord blood CD34 cells grown for 7 days on the developed HSC recipe shown in Table 1 (FIG. 9A) or on the control recipe (SCF and TPO) (FIG. 9B). Cells were stained with antibodies for Lin, CD34, CD38, CD45RA, CD90 and FVS700 a live and dead marker, to exclude dead cells from the analysis,

FIG. 10 shows results from an HD-DoE model of a 12-factor experiment optimized for maximum expression of CRHBP. The upper section of the model shows the prediction of expression level of pre-selected 53 genes when optimized for CRHBP. The lower section of the model shows the effectors that were tested in this model and their contribution to maximum expression of CRHBP. The value column refers to required concentration of each effector to mimic the model.

### DETAILED DESCRIPTION OF THE INVENTION

Described herein are methodologies and compositions that allow for maintenance and expansion of human CD34+ hematopoietic stem cells (HSCs) under chemically-defined culture conditions using a small molecule based approach. As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to maintain and expand HSCs in a very short amount of time. The optimized culture media was further validated by a factor criticality analysis, which examined the effects of eliminating individual agonist or antagonist agents, as described in Example 2. Flow cytometry analysis further confirmed the phenotype of the cells generated by the differentiation protocol, as described in Example 3. A subprotocol for maintaining long term HSCs (LT-HSCs) also was determined, as described in Example 4.

Various aspects of the invention are described in further detail in the following subsections.

### I. Cells

The starting cells used in the cultures of the disclosure are human CD34+ hematopoietic stem cells. As used herein, the term “hematopoietic stem cell” (abbreviated as HSC) refers to a stem cell that has the capacity to differentiate into a variety of different hematopoietic cell types. CD34 is a transmembrane phosphoglycoprotein that has been established in the art as a surface marker for HSCs. Human HSCs are readily obtainable from available sources, including human umbilical cord blood and adult bone marrow. HSCs include both long term HSCs (LT-HSCs) and short term HSCs (ST-HSCs).

Long term HSCs (LT-HSCs) are HSCs that are found in the bone marrow or cord blood that, through a process of asymmetric cell division, can self-renew to sustain the stem cell pool or differentiate into short-term HSCs (ST-HSCs) or lineage-restricted progenitors that undergo extensive proliferation and differentiation to produce terminally differentiated cells of the blood lineage. It is believed that LT-HSCs are enriched on the fraction of Lin-CD34+CD38−CD45RA-CD90+ cells. LT-HSCs are quiescent and slow to divide in culture, taking up to 80 hours to first cell division (Cheung and Rando (2013) Nat. Rev. Mol. Cell Biol. 14:329-340). In contrast, short term HSCs (ST-HSCs) by definition have limited self-renewal capacity, generally described as giving rise to lymphohernatopoiesis for 4-12 weeks before senescence.

In an embodiment, the HSCs express CD34 (CD34+), In an embodiment, the HSCs lack expression of the marker Lineage (Lin−). In an embodiment, the HSCs lack expression of CD38 (CD38−). In an embodiment, the HSCs lack expression of CD45RA (CD45RA−). In an embodiment, the HSCs express CD90 (CD90+). In an embodiment, the HSCs are Lin-CD34+CD38-CD45RA-CD90+ cells.

In embodiments, the HSCs express one or more genes associated with the HSC phenotype (also referred to herein as HSC-associated genetic markers), non-limiting examples of which include CHRBP, Mecom, Meg3, HOPX, LMO2, CD34, TAL1 and GATA2.

In an embodiment, the LT-HSCs are CD34+. In an embodiment, the LT-HSCs are CD34+ and also express CRHBP, HOPX and LMO2.

### II. Culture Media Components

The method of the disclosure for maintaining and/or expanding CD34+ HSCs comprise culturing human CD34+ HSCs in a culture media comprising specific agonist and/or antagonists of cellular receptors and/or signaling pathways.

As described in Example 1, a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor was sufficient to maintain and expand CD34+ HSCs in as little as six days.

Additionally, as described in Example 4, a culture media comprising a TGFβ pathway agonist, a bioactive phospholipid, an aryl hydrocarbon receptor (AhR) agonist and a histone deacetylase (HDAC) inhibitor was developed for maintaining LT-HSCs. This culture media imparts only a low proliferative capacity to the LT-HSCs due to the lack of c-kit ligand and TPOR agonist.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

C-kit ligands, TPOR agonists, TGFβ pathway agonists, antioxidants, bioactive phospholipids, aryl hydrocarbon receptor (AhR) agonists, Notch agonists and histone deacetylase (HDAC) inhibitors are known in the art and commercially available. They are used in the culture media at a concentration effective to achieve the desired outcome, e.g., maintenance and/or expansion of HSCs expressing markers of interest. Non-limiting examples of suitable agonist and antagonists agents, and effective concentration ranges, are described further below.

C-kit ligands include agents, molecules, compounds or substances that bind to the c-kit receptor (CD117). In an embodiment, the c-kit ligand is stem cell factor (SCF). In an embodiment, SCF is present in the media at a concentration range of 5-25 ng/ml. In an embodiment, SCF is present in the media at a concentration of 10 ng/ml.

TPOR agonists include agents, molecules, compounds or substances that agonize the thrombopoietin receptor (TPOR). In an embodiment, the TPOR agonist is thrombopoietin (TPO). In other embodiments, the TPOR agonist is eltrombopag, TA-316, TPO agonist 1, avatrombopag or lusutrombopag. In an embodiment, the TPOR agonist is TPO, which is present in the media at a concentration range of 50-150 ng/ml. In an embodiment, the TPOR agonist is TPO, which is present in the media at a concentration of 100 ng/ml.

Agonists of the TGFβ pathway include agents, molecules, compounds, or substances capable of stimulating (activating) the TGFβ signaling pathway. In an embodiment, the TGFβ pathway agonist is Activin A. In another embodiment, the TGFβ pathway agonist is alantolactone. In an embodiment, the TGFβ pathway agonist is Activin A, which is present in the media at a concentration range of 10-30 ng/ml. In an embodiment, the TGFβ pathway agonist is Activin A, which is present in the media at a concentration of 20 ng/ml.

Antioxidants include agents, molecules, compounds, or substances that prevent or slow the damage to cells caused by free radicals. In an embodiment, the antioxidant is vitamin C. In one embodiment, the vitamin C is L-ascorbic acid phosphate sesquimagnesium salt hydrate, a stable form of vitamin C (Vit. C). In other embodiments, the antioxidant is ascorbic acid, glutathione, ebeselen, N-acetyl-L-cysteine or α-tocopherol. In an embodiment, the antioxidant is vitamin C, which is present in the media at a concentration range of 50-150 uM. In an embodiment, the antioxidant is vitamin C, which is present in the media at a concentration of 100 uM.

In an embodiment, the bioactive phospholipid is lysophosphatidic acid (LPA). In other embodiments, the bioactive phospholipid is sphingosine-1-phosphate (SIP), ceramide-1-phosphate (C1P) or lysophosphatidylcholine (LPC). In an embodiment, the bioactive phospholipid is LPA, which is present in the media at a concentration range of 100-300 uM. In an embodiment, the bioactive phospholipid is LPA, which is present in the media at a concentration of 200 uM.

Agonists of AhR include agents, molecules, compounds, or substances capable of stimulating (activating) the AhR signaling pathway. In an embodiment, the AhR agonist is 6-Formylindolo[3,2-b]carbazole (FICZ). In other embodiments, the AhR agonist is Norisoboldine, Pifithrin-α hydrobromide, MeBIO, ITE or 10-C1-BBQ. In an embodiment, the AhR agonist is FICZ, which is present in the media at a concentration range of 250-750 nM. In an embodiment, the AhR agonist is FICZ, which is present in the media at a concentration of 500 nM.

Agonists of Notch include agents, molecules, compounds, or substances capable of stimulating (activating) the Notch signaling pathway. In an embodiment, the Notch agonist is Yhhu 3792. In other embodiments, the Notch agonist is Jagged 1-2 or DLL1-4. In an embodiment, the Notch agonist is Yhhu 3792, which is present in the media at a concentration range of 500-1000 nM. In an embodiment, the Notch agonist is Yhhu 3792, which is present in the media at a concentration of 750 nM.

In an embodiment, the HDAC inhibitor is valproic acid (VPA). In other embodiments, the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1. In an embodiment, the HDAC inhibitor is VPA, which is present in the media at a concentration range of 100-200 uM. In an embodiment, the HDAC inhibitor is VPA, which is present in the media at a concentration of 150 uM.

In an embodiment, the method of expanding or maintaining human CD34+ hematopoietic stem cells (HSCs) comprises culturing human CD34+ HSCs in a culture media comprising Stem Cell Factor (SCF), thrombopoietin (TPO), Activin A, Vitamin C, lysophosphatidic acid (LPA), 6-Formylindolo[3,2-b]carbazole (FICZ), Yhhu 3792 and valproic acid (VPA). In an embodiment, these agents are present in the media at a concentration range as set forth above. In an embodiment, SCF is present at a concentration of 10 ng/ml, TPO is present at a concentration of 100 ng/ml, Activin A is present at a concentration of 20 ng/ml, Vitamin C is present at a concentration of 100 uM, LPA is present at a concentration of 200 nM, FICZ is present at a concentration of 500 nM, Yhhu 3792 is present at a concentration of 750 nM and VPA is present at a concentration of 150 uM.

### III. Culture Conditions

In combination with the chemically-defined and optimized culture media described in subsection II above, the methods of maintaining or expanding CD34+ HSCs of the disclosure utilize standard culture conditions established in the art for cell culture. For example, cells can be cultured at 37° C. and under 5% 02 and 5% CO₂ conditions. A basal media can be used as the starting media to which supplemental agents can be added. For example, in an embodiment, the commercially available StemSpan™ SFEM II media is used as basal media. Cells can be cultured in standard culture vessels or plates, such as culture dishes, culture flasks or 96-well plates.

The starting CD34+ HSCs can be obtained by methodologies established in the art. Sources of human CD34+ HSCs include umbilical cord blood and bone marrow. CD34+ HSC can be obtained, for example, by standard magnetic enrichment.

To expand CD34+ HSCs, the cells are cultured in the optimized culture media for sufficient time to expand the CD34+ HSC population (i.e., increase the number of CD34+ HSCs in the culture). As described in the Examples, it has been discovered that culture of CD34+ HSCs in the optimized culture media for as little as six days was sufficient for CD34+ HSC expansion and expression of desired cellular markers.

In various embodiments, the CD34+ HSCs are cultured in the optimized culture media for sufficient time to increase the expression of at least one, and preferably a plurality of, HSC-associated genetic markers. Non-limiting examples of suitable HSC-associated genetic markers include CHRBP, Mecom, Meg3, HOPX, LMO2, CD34, TAL1 and GATA2. In embodiments, cells are cultured for sufficient time to increase the expression levels of at least two, at least three, at least four, at least five, at least six, at least seven or at least eight HSC-associated genetic markers. In an embodiment, cells are cultured for sufficient time to increase the expression level of at least one HSC-associated genetic marker (e.g., CHRBP) by at least 10%, 20%, 30%, 40%, 50%, 60%, 70%, 80%, 90% or 100% as compared to the starting cell population. The level of expression of genetic markers in the cultured HSCs can be measured by techniques available in the art (e.g., RNAseq analysis).

In an embodiment, cells are cultured for at least 3 days, at least 4 days, at least 5 days, at least 6 days, at least 7 days, at least 10 days, at least 1 week, at least 2 weeks, at least 3 weeks, at least 4 weeks or longer.

The culture media typically is changed regularly to fresh media. For example, in one embodiment, media is changed every 72 hours.

### IV. Uses

The methods and compositions of the disclosure for maintaining and expanding HSCs allow for efficient and robust availability of these cell populations for a variety of uses. For example, the methods and compositions can be used in the study of HSC development and differentiation, including biology to assist in the understanding of hematopoietic-related diseases and disorders. For example, the HSCs (e.g., LT-HSCs) generated using the methods of the disclosure can be further purified according to methods established in the art using agents that bind to surface markers expressed on the cells.

Additionally, HSCs maintained and/or expanded according to the methods of the disclosure are contemplated for use in the treatment of various hematopoietic diseases and disorders, through delivery of the cells to a subject having the disease or disorder (e.g., HSC transplantation). Examples of hematopoietic diseases and disorders include, but are not limited to, cancers such as leukemias and lymphomas, blood disorders and autoimmune disorders.

### V. Compositions

In other aspects, the disclosure provides compositions related to the methods of maintaining and expanding HSCs, including culture media and isolated cell cultures.

In one aspect, the disclosure provides a culture media for maintaining or expanding human CD34+ hematopoietic stem cells (HSCs) comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor. In another aspect, the disclosure provides a culture media for maintaining human CD34+ long term hematopoietic stem cells (LT-HSCs) comprising a TGFβ pathway agonist, a bioactive phospholipid, an aryl hydrocarbon receptor (AhR) agonist and a histone deacetylase (HDAC) inhibitor. Non-limiting examples of suitable agents, and concentrations therefor, include those described in subsection II above.

In another aspect, the disclosure provides an isolated cell culture of human CD34+ hematopoietic stem cells (HSCs), the culture comprising: human CD34+ HSCs cultured in a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor. In another aspect, the disclosure provides an isolated cell culture of human CD34+ long term hematopoietic stem cells (LT-HSCs), the culture comprising: human CD34+LT-HSCs cultured in a culture media comprising a TGFβ pathway agonist, a bioactive phospholipid, an aryl hydrocarbon receptor (AhR) agonist and a histone deacetylase (HDAC) inhibitor. Non-limiting examples of suitable agents, and concentrations therefor, include those described in subsection II above.

The present invention is further illustrated by the following examples, which should not be construed as further limiting. The contents of figures and all references, patents and published patent applications cited throughout this application are expressly incorporated herein by reference.

### Examples

### Example 1: Culture Protocol Development for Expansion and Maintenance of Hematopoietic Stem Cells

In this example, a culture media recipe for expansion and maintenance of HSCs was developed. A recipe for expansion and maintenance of hematopoietic stem cells was developed that can be used to culture CD34⁺ cells, for example from cord blood, and expand a population of cells referred to in the art as long-term HSC (LT-HSC) identified as Lin⁻CD34⁺CD38⁻CD45RA⁻CD90⁺.

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for expansion and maintenance of HSCs.

Specifically, to develop the HSC recipe, the impact of agonists and antagonists of multiple signaling pathways (herein called effectors) were tested on expression of two sets of 53 pre-selected genes after a 3-day treatment. Specific genes in the measured subset are highly enriched in HSC and include CRHBP, MLLT3, MEG3, MECOM, HOXA5 and GATA2. The effectors chosen include small molecules or proteins, some of which are used during in vitro expansion of HSC.

To test the effectors, experiments with at least 8 factors were designed that can assess the response of cells to 48 or more different combinations of effectors in a range of concentrations. To analyze the models, we focused on expression of genes that are either expressed or enriched in primitive HSC including CRHBP, GATA2, MLLT3 and MEG3. The impact of each effector on gene expression level is defined by a parameter called factor contribution that is calculated for each effector during the modeling. All the experiments were conducted using a CD34⁺ population of cells isolated from cord blood.

To identify the recipe for HSC expansion, cells were treated with various effectors for 3 days in media containing stem cell factor (SCF) and thrombopoietin (TPO) and the gene expression of cells was modeled. One model specifically, showed promising results on upregulation of GATA2, CRHBP, HOXA5 and MECOM and MEG3. Additionally, on this model we observed NOTCH1 upregulation and MPO downregulation. MPO is a myeloid marker highly expressed in neutrophils and other blood progenitor cells. Previous studies have shown that NOTCH1 activation increases HSC self-renewal in vivo (Stier et al. (2002) Blood 99:2369-78).

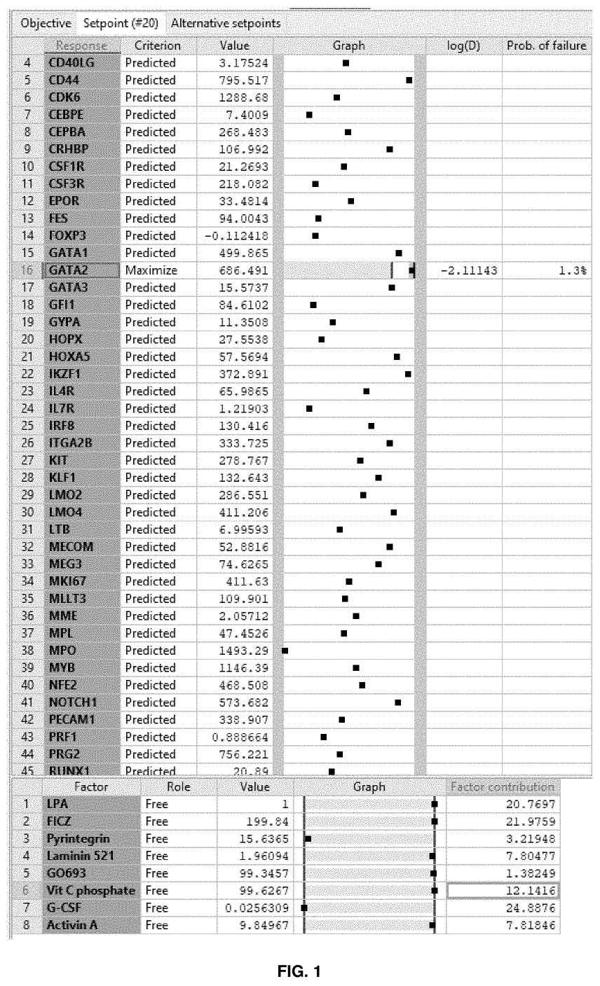

As shown in the results summarized in FIG. 1, one model specifically showed promising results when optimized for maximum expression of GATA2. This model tested 8 factors: LPA, FICZ, Pyrintegrin, Laminin521, G0693, Vit.C, G-SCF, and activin A. Three of the effectors, (LPA, FICZ and Vit.C) showed positive impact on expression of genes of interest with 20, 21, and 12 factor contributions, respectively. CD44 is a receptor highly expressed in cells undergoing endothelial to hematopoietic transition, the process that is crucial for HSC generation (Oatley et al. (2020)Nat Commun. 11:586). Interestingly, we observed maximum levels of CD44 when we optimized for GATA2.

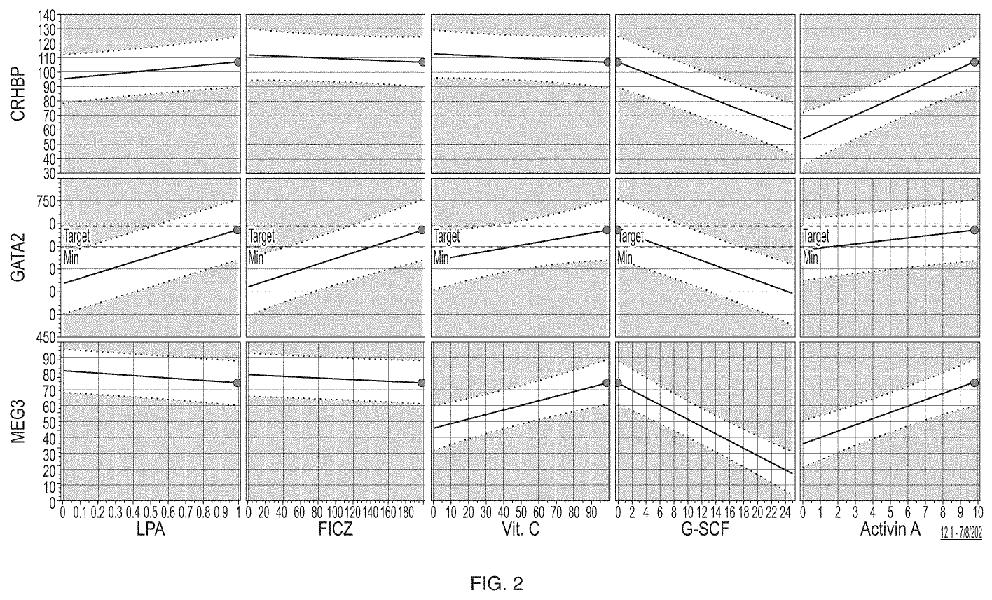

MEG3 is highly expressed in mouse and human HSCs and is strongly down-regulated in early progenitors (Sommerkamp et el. (2019) Sci Rep 9:2110). In order to bring MEG3 to its maximal expression level, we next optimized the model for maximum expression of MEG3. Two effectors with significant positive effect on expression of Meg3 were identified including Activin A, and Vit. C with 23 and 16 factor contributions, respectively (FIG. 2). Despite having a modest factor contribution for GATA2, Activin A was one of the factors that was critical for many other HSC enriched genes including MEG3, HOXA5 and MECOM, therefore Activin A is included in the recipe.

To further improve the recipe for HSC maintenance and expansion, additional HD-DoE experiments were performed. These data generated additional gene regulatory models that was used for preparation of the expansion protocol. The goal was to evaluate further factors to possibly increase the complexity of the signaling inputs to attain effective fate control. As previously, we focused on expression of HSC enriched genes mentioned earlier. An 8-factor experiment was performed, testing tropoelastin, lipid mixture, propionate, biotin, geltrex, N-acetyl cysteine, prostaglandin E2, and VPA. Similar to the previous experiment, CD34⁺ cells were treated with 48 conditions of combinations of the factors for 3 days and gene expression analysis and modeling was performed.

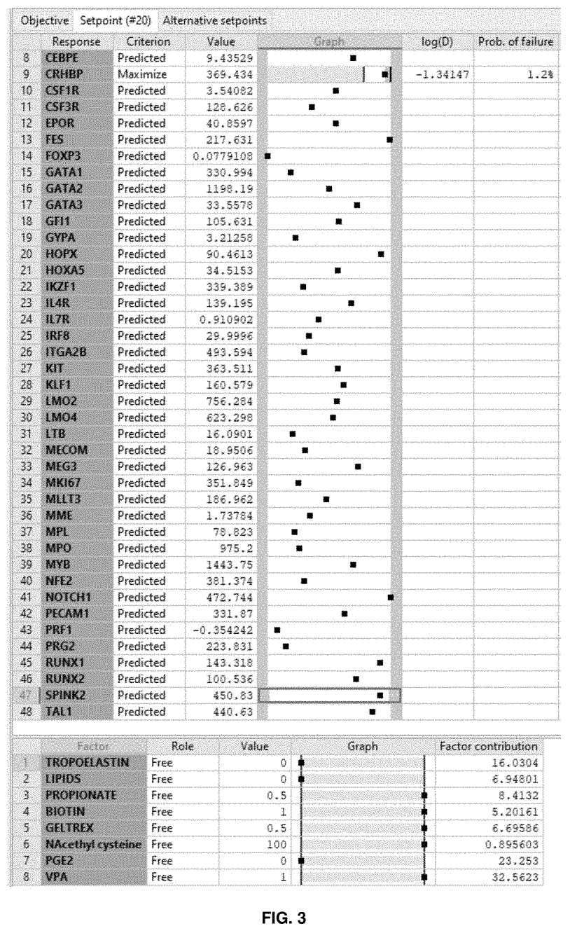

As shown in the results summarized in FIG. 3, when optimized for CRHBP, one factor, VPA (histone deacetylase inhibitor) had significant positive impact with factor contribution of 32. Propionate, biotin and geltrex also had positive impacts but their factor contribution was less than 10 (8, 5 and 6 respectively) and N-acetyl cysteine had <1 positive factor contribution.

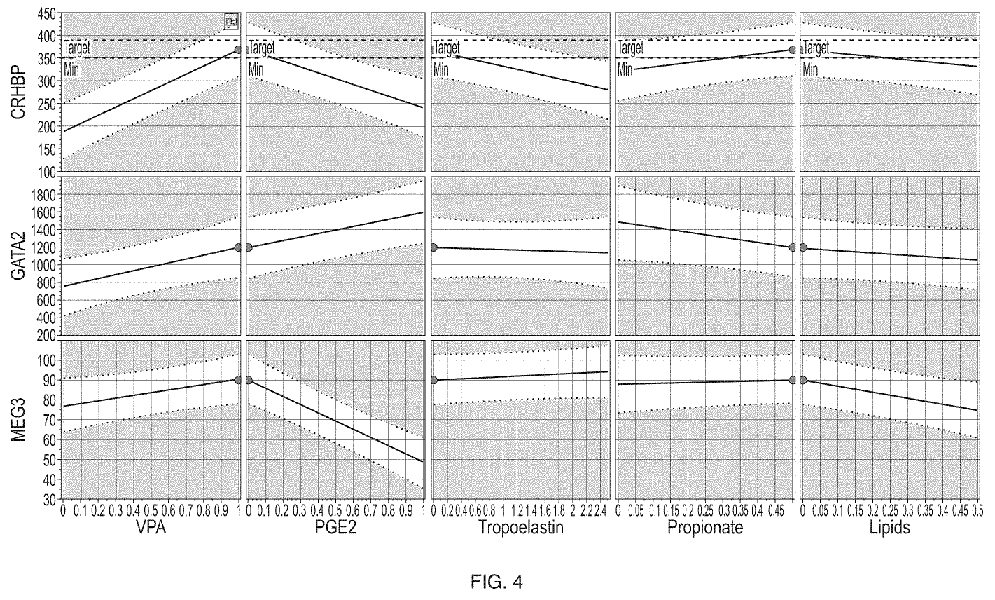

Optimization for CRHBP showed promising results on upregulation of HOPX, (a gene required for HSC stemness, (Lin et al. (2020) Oncogene 39:5112-5123), NOTCH1, SPINK2 (a gene enriched in nascent HSC) and RUNX1 (a gene required for HSC generation during embryogenesis (North et al. (2004) Stem Cells 22:158-68). Because propionate, biotin and geltrex showed low factor contributions in this context and it might downregulate other HSC enriched genes, we excluded these factors, maintaining VPA. When the same experiment was optimized for maximum expression of GATA2 three effectors were identified with significant positive impact on expression of GATA2 which include VPA, prostaglandin E2, and tropoelastin with 33, 25 and 17 factor contributions (FIG. 4). Because tropoelastin and prostaglandin E2 treatment downregulates other HSC enriched genes, we decided to exclude these factors but maintained VPA.

To further improve the recipe for HSC maintenance and expansion, additional HD-DoE experiments were performed. Another 8-factor experiment was performed consisting of LPA, Yhhu 3792, fibronectin, TPCA-1, GW9662, Z-vad-fmk, VEGF and activin A. Similarly to the previous experiment, CD34⁺ cells were treated with 48 conditions of combinations of the factors for 3 days and then gene expression analysis experiments were conducted.

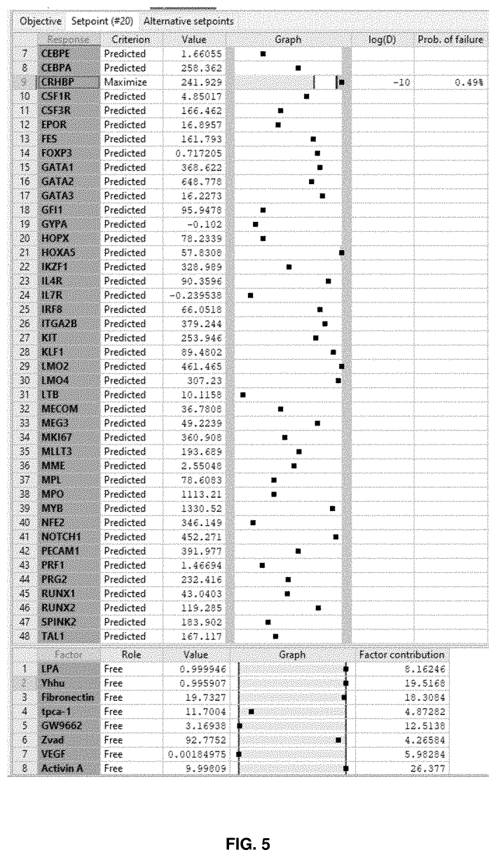

As shown in the results summarized in FIG. 5, when optimized for CRHBP, one factor, Activin A (agonist of the TGF beta family) had significant positive impact with factor contribution of 26, confirming our previous results. LPA, Yhhu 3792 and fibronectin also had positive impacts with factor contribution of 8, 19 and 18 respectively. Conditions required for CRHBP optimization induced other HSC related genes such as HOXA5, NOTCH1, MEG3 as well. LMO2 and LMO4 were also upregulated in this condition. Both genes are enriched in HSCs, however, expressed by other blood progenitors as well. In order to decrease the complexity of the recipe, we decided include only Yhhu 3792; a Notch signaling agonist.

Considering all the models analyzed, based on predicted conditions that maximize expression of HSC enriched genes such as GATA2, CRHBP, HOXA5, MLLT3, MEG3 and NOTCH1 we developed a complex recipe for HSC maintenance and expansion was developed that is composed of 8 effectors, as shown below in Table 1:

TABLE 1
HSC Maintenance and Expansion Culture Media Recipe

| Effectors | Role | Concentration | |
|---|---|---|---|
| Stem cell factor (SCF) | C-kit ligand | 10 | ng/mL |
| Thrombopoietin (TPO) | TPOR ligand | 100 | ng/mL |
| Activin A | TGFb pathway agonist | 20 | ng/mL |
| L-ascorbic acid phosphate | Antioxidant | 100 | uM |
| sesquimagnesium salt | | | |
| hydrate (Vit. C) | | | |
| Lysophosphatidic aid | Bioactive phospholipid | 200 | nM |
| (LPA) | | | |
| 6-Formylindolo[3,2- | aryl hydrocarbon receptor | 500 | nM |
| b]carbazole (FICZ) | (AhR) agonist | | |
| Yhhu 3792 | Notch agonist | 750 | nM |
| Valproic acid (VPA) | Histone deacetylase | 150 | uM |
| | inhibitor | | |

### Example 2: Factor Criticality Analysis of HSC Maintenance and Expansion Media

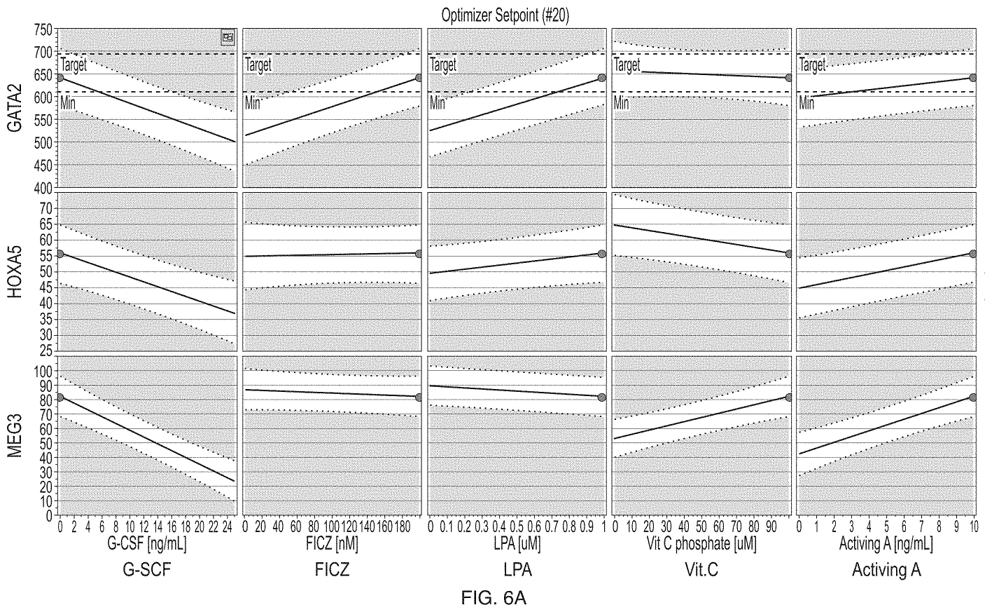

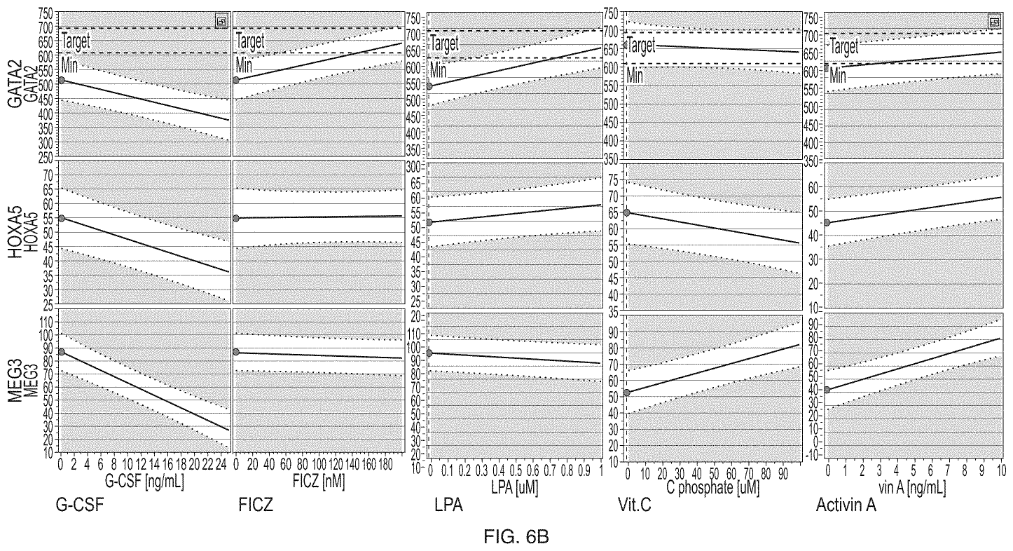

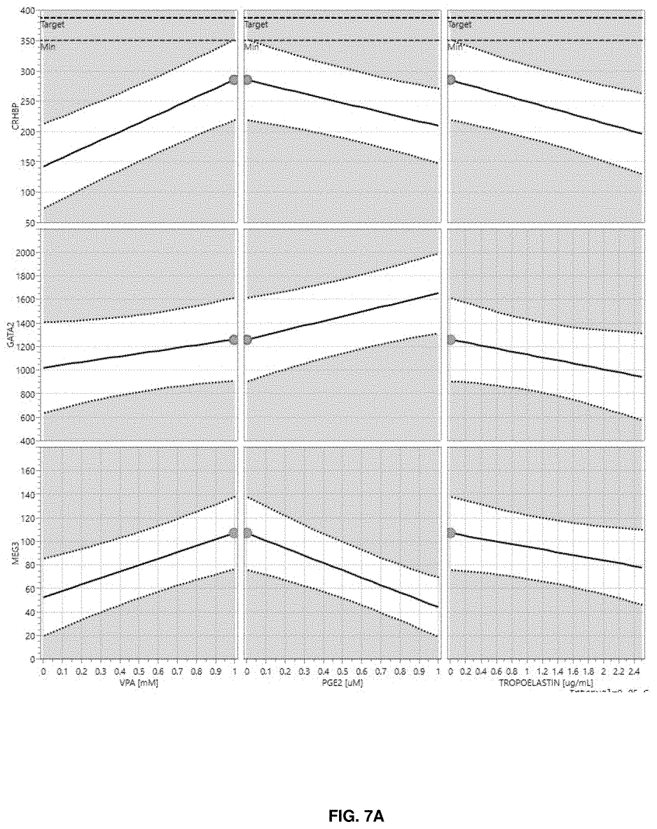

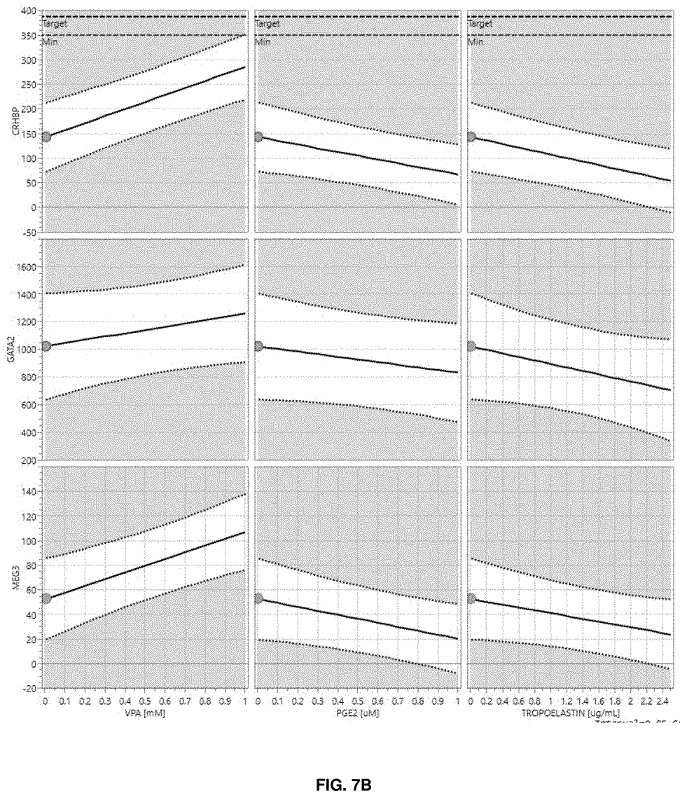

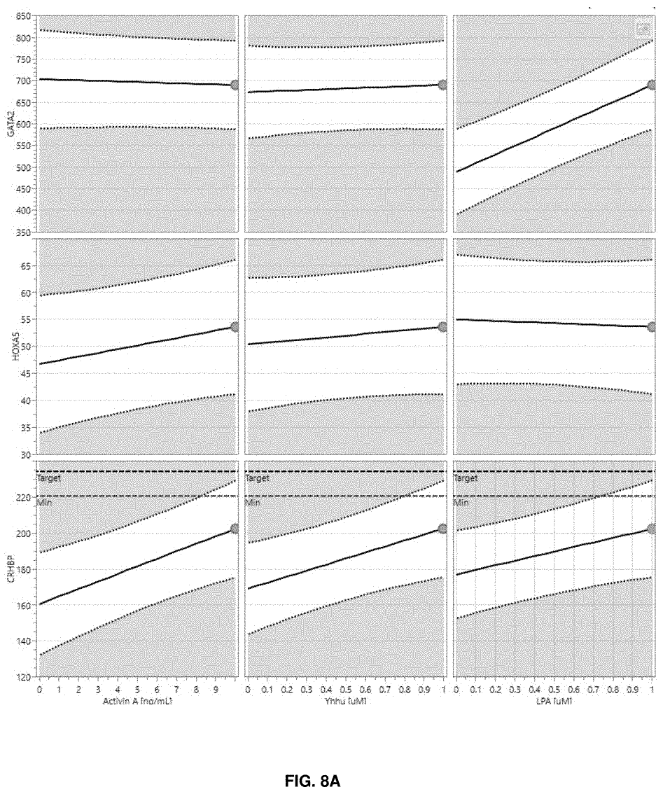

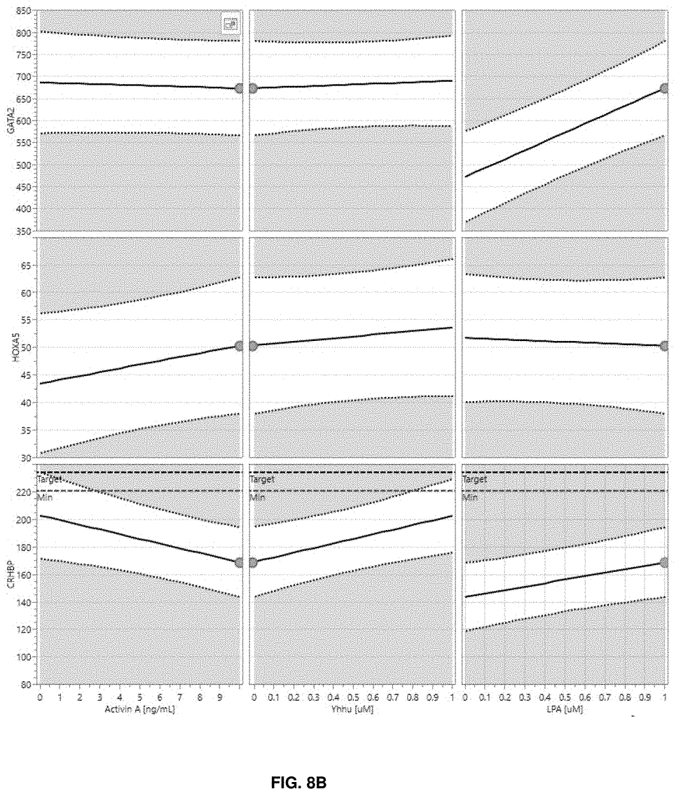

In this example, to assess the impact of elimination of each validated factor identified as described in Example 1, dynamic profile analysis was used and compared the expression level of genes of interest in the absence of each finalized factor while others are kept present. Since the expression levels of genes of interest revealed whether the desired outcome was reachable, this factor criticality analysis revealed the extent of importance of each input effector. The results of the criticality analysis for the factors FICZ, LPA, Vit. C and Activin A are shown in FIG. 6A-B. The results of the criticality analysis for VPA are shown in FIG. 7A-B. The results of the criticality analysis for Yhhu 3792 are shown in and FIG. 8A-B.

For the HSC expansion recipe, each of the six finalized factors were removed while other five factors were present and the expression levels of GATA2, HOXA5, CRHBP was assessed compared to the presence of all five factors. Because SCF and TPO are cytokines impacting cell growth and survival, our recipe included these cytokines as well. When FICZ was removed, values for GATA2 expression decreased from 642 to 512, while HOXA5 and MEG3 expression was unchanged. Absence of LPA resulted in reduced expression of GATA2, a decrease from 644 to 525. Removal of Vit.C decreased the level of MEG3 from 82 to 53. Activin A is a critical factor for MEG3, since its removal decreases MEG3 levels from 81 to 40. Activin A also has a positive impact on GATA2 and HOXA5 but at a lower extent.

VPA is a histone deacetylase inhibitor that has been used to expand HSC (Papa et al. (2018) Blood Adv. 2:2766-2779). In general, this class of drugs promotes chromatin opening, which can result in either up-regulation or repression of genes. VPA was included in the HSC recipe because removal of VPA decreased levels of CRHBP from 285 to 138. MEG3 levels were reduced to half when VPA was removed (from 106 to 52). Finally, GATA2 levels were also reduced upon VPA removal however to a lower extent.

The function of NOTCH pathway on hematopoiesis is complex and has some controversies (see e.g., Huang et al. (2021) Front. Cell Biol. DOI:10.3389/fcell.2020.606448). However, previous studies have shown that NOTCH stimulation by Delta-1 enhances the repopulation ability of human CD34⁺CD38⁻ cells. Yhhu 3792 is a new NOTCH agonist first described in 2018 (Lu et al. (2018) Stem Cells 36:1273-1285). The experiments herein revealed that Yhhu 3792 is a promoter of the HSC phenotype. Removal of Yhhu 3792 decreased levels of CRHBP from 202 to 169. The factor criticality analysis demonstrates that removal of any of the inputs are not critical for performance. This is expected due to the high complexity and number of the signaling inputs.

### Example 3: Flow Cytometry Validation of Culture Media for Maintenance and Expansion of Cord Blood HSC

To further validate the HSC culture recipe described in Example 1, CD34+ cord blood derived cells were grown for 7 days in media comprising the ingredients shown in Table 1 and flow cytometry analysis was used to assess markers of the LT-HSC state. Markers tested included Lineage (CD3, CD14, CD16, CD19, CD20, CD56), CD34, CD38, CD45RA and CD90. Additionally, cells were grown in media with SCF and TPO as a control, commonly used by others to promote HSC expansion. Other commonly used cytokines to expand HSC such as IL3, G-CSF and IL6 were not used, since our studies showed an induction of a myeloid bias when CD34+ cells were cultured in the presence of these cytokines. 100,000 cells were plated for each condition.

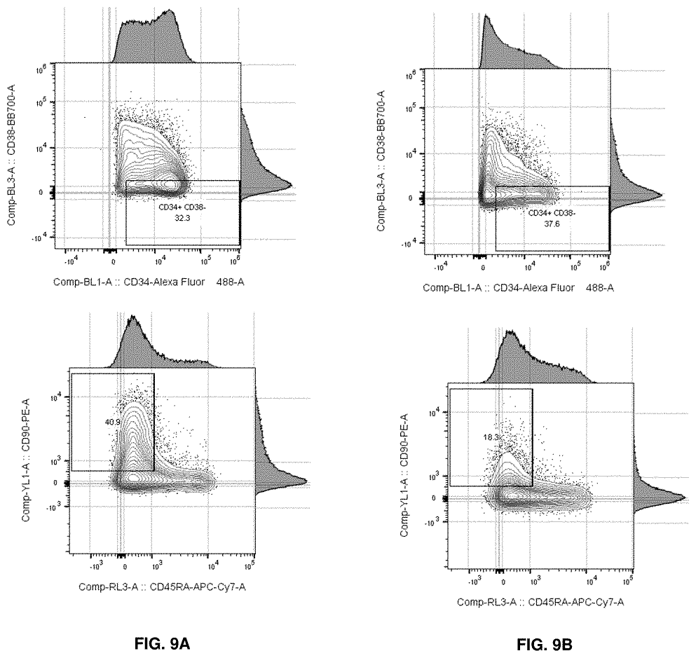

After growing the cells for 7 days, flow cytometry analysis was performed. The immunophenotype of LT-HSC, namely Lin⁻CD34⁺CD38⁻CD45RA⁻CD90⁺ was evaluated. The results are shown in FIG. 9A-B. Flow cytometry analysis confirmed the efficiency of the HSC recipe to promote HSC expansion and maintenance. In conditions supplying SCF+TPO (control), 18.3% of the cells were CD45RA″ and CD90⁺ (18.3% of the parent gate (CD34⁺CD38″)). In contrast, the complex media recipe as described in Example 1 resulted in 40.9% of cells being CD45RA″ and CD90⁺ (40.9% of the parent gate (CD34⁺CD38″)).

### Example 4: Development of Conditions for LT-HSC Quiescent State Control

The aforementioned conditions described in Example 1 that generated improved numbers of LT-HSC were done in the context of continuous expansion. However, the feature set of the LT-HSC is invariably linked to quiescence; thus, at a critical level, the resulting expanded CD34+ cells are not identical to the resident LT-HSC population in marrow. For that reason, additional HD-DOE experiments were performed in basal media without SCF and TPO. On these experiments, the design was composed of 12 factors, including the factors on Table 1, plus SCF, TPO, FLT-3L, IGFII, Yoda-1 and cytosporin B.

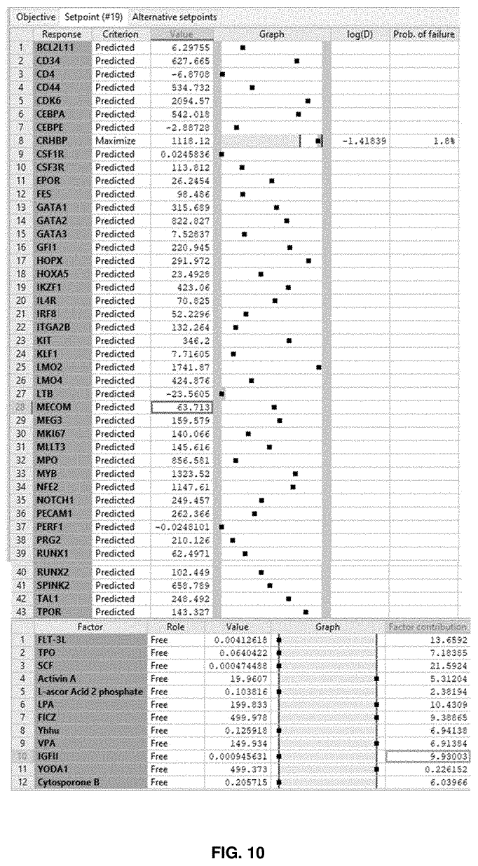

One model, the results of which are summarized in FIG. 10, specifically showed promising results on upregulation of CRHBP, HOPX, CD34, LMO2. Levels of CRHBP were extremely high on this experiment, reaching the level of 1118. Interestingly, many factors present in Table 1 were required to induce maximal CRHBP expression in the absence of cytokines, confirming the importance of these factors for cell stemness.

The best conditions to induce maximal CRHBP levels are shown below in Table 2.

TABLE 2

| Effectors | Role | Concentration | |
|---|---|---|---|
| Activin A | TGFb pathway agonist | 20 | ng/mL |
| Lysophosphatidic aid | Bioactive phospholipid | 200 | nM |
| (LPA) | | | |
| 6-Formylindolo[3,2- | aryl hydrocarbon receptor | 500 | nM |
| b]carbazole (FICZ) | (AhR) agonist | | |
| Valproic acid (VPA) | Histone deacetylase | 150 | uM |
| | inhibitor | | |

In order to compare the media in Table 2 with traditional methods for HSC culture and expansion (SCF+TPO), through mathematical modeling we create predictions of genes levels related to the stem cell phenotype, as well as lineage specific genes, when cultured in either traditional media (SCF+TPO) or the media of Table 2. These predicted gene levels are summarized below in Table 3:

TABLE 3

| | Normalized Gene Expression | | | | |
|---|---|---|---|---|---|
| | | | | | Relative |
| | | | | SCF + | Expression |
| | | | | TPO + | (Table2/ |
| | | SCF + | Table 2 | Table2 | SCF + |
| GENE | MARKER | TPO | Recipe | recipe | TPO) |
| CHRBP | LT-HSC | 186 | 1113 | 381 | 5.98 |
| Mecom | LT-HSC | 30 | 34 | 59 | 1.1 |
| Meg3 | LT-HSC | 1 | 187 | 117 | 187 |
| HOPX | LT-HSC | 139 | 325 | 91 | 2.33 |
| LMO2 | LT-HSC | 494 | 1784 | 1084 | 3.61 |
| CD34 | LT-HSC + | 222 | 676 | 471 | 3.04 |
| | HSC | | | | |
| TAL1 | HSC | 110 | 293 | 245 | 2.66 |
| | commitment | | | | |
| GATA2 | HSC + | 619 | 639 | 412 | 1.03 |
| | myeloid | | | | |
| | (early) | | | | |
| KLF1 | Erythroid | 48 | 6 | 37 | 0.125 |
| MPO | Myeloid | 3137 | 704 | 1164 | 0.22 |
| | lineages | | | | |
| MKI67 | proliferation | 249 | 53 | 155 | 0.21 |
| BCL2L11 | apoptosis | 11 | 21 | 33 | 1.9 |

As expected, because the Table 2 recipe does not include SCF and TPO, proliferation (MKI67 level) was lower when cells were grown in this recipe. However, stem cell related genes such as CRHBP, MEG3, HOPX and CD34 were highly expressed in media from Table 2 as compared to SCF+TPO condition. On the other hand, KLF1 and MPO, two markers of lineage committed cells, were highly expressed in SCF+TPO conditions.

### EQUIVALENTS

Those skilled in the art will recognize, or be able to ascertain using no more than routine experimentation, many equivalents of the specific embodiments of the invention described herein. Such equivalents are intended to be encompassed by the following claims

1. A method of expanding or maintaining human CD34+ hematopoietic stem cells (HSCs) comprising culturing human CD34+ HSCs in a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor.

2. The method of claim 1, wherein the c-kit ligand is stem cell factor (SCF).

3. The method of claim 1, wherein the TPOR agonist is thrombopoietin (TPO).

4. The method of claim 1, wherein the TPOR agonist is eltrombopag, TA-316, TPO agonist 1, avatrombopag or lusutrombopag.

5. The method of claim 1, wherein the TGFβ pathway agonist is Activin A.

6. The method of claim 1, wherein the TGFβ pathway agonist is alantolactone.

7. The method of claim 1, wherein the antioxidant is vitamin C.

8. The method of claim 1, wherein the antioxidant is ascorbic acid, glutathione, ebeselen, N-acetyl-L-cysteine or α-tocopherol.

9. The method of claim 1, wherein the bioactive phospholipid is lysophosphatidic acid (LPA).

10. The method of claim 1, wherein the bioactive phospholipid is sphingosine-1-phosphage (SIP), ceramide-1-phosphate (C1P) or lysophosphatidylcholine (LPC).

11. The method of claim 1, wherein the AhR agonist is 6-Formylindolo[3,2-b]carbazole (FICZ).

12. The method of claim 1, wherein the AhR agonist is Norisoboldine, Pifithrin-α hydrobromide, MeBIO, ITE or 10-C1-BBQ.

13. The method of claim 1, wherein the Notch agonist is Yhhu 3792.

14. The method of claim 1, wherein the Notch agonist is Jagged 1-2 or DLL1-4.

15. The method of claim 1, wherein the HDAC inhibitor is valproic acid (VPA).

16. The method of claim 1, wherein the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1.

17. The method of claim 1, wherein the CD34+ HSCs are from umbilical cord blood.

18. The method of claim 1, wherein the CD34+ HSCs are from bone marrow.

19. The method of claim 1, wherein the CD34+ HSCs are cultured for at least six days.

20. The method of claim 1, wherein the CD34+ HSCs have a phenotype of Lin-CD34+CD38-CD45RA-CD90+.

21. A method of expanding or maintaining human CD34+ hematopoietic stem cells (HSCs) comprising culturing human CD34+ HSCs in a culture media comprising Stem Cell Factor (SCF), thrombopoietin (TPO), Activin A, Vitamin C, lysophosphatidic acid (LPA), 6-Formylindolo[3,2-b]carbazole (FICZ), Yhhu 3792 and valproic acid (VPA).

22. The method of claim 21, wherein SCF is present at a concentration of 10 ng/ml, TPO is present at a concentration of 100 ng/ml, Activin A is present at a concentration of 20 ng/ml, Vitamin C is present at a concentration of 100 uM, LPA is present at a concentration of 200 nM, FICZ is present at a concentration of 500 nM, Yhhu 3792 is present at a concentration of 750 nM and VPA is present at a concentration of 150 uM.

23. A culture media for expanding or maintaining human CD34+ hematopoietic stem cells (HSCs) comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor.

24. The culture media of claim 23, wherein the c-kit ligand is stem cell factor (SCF).

25. The culture media of claim 23, wherein the TPOR agonist is thrombopoietin (TPO).

26. The culture media of claim 23, wherein the TPOR agonist is eltrombopag, TA-316, TPO agonist 1, avatrombopag or lusutrombopag.

27. The culture media of claim 23, wherein the TGFβ pathway agonist is Activin A.

28. The culture media of claim 23, wherein the TGFβ pathway agonist is alantolactone.

29. The culture media of claim 23, wherein the antioxidant is vitamin C.

30. The culture media of claim 23, wherein the antioxidant is ascorbic acid, glutathione, ebeselen, N-acetyl-L-cysteine or α-tocopherol.

31. The culture media of claim 23, wherein the bioactive phospholipid is lysophosphatidic acid (LPA).

32. The culture media of claim 23, wherein the bioactive phospholipid is sphingosine-1-phosphage (S1P), ceramide-1-phosphate (C1P) or lysophosphatidylcholine (LPC).

33. The culture media of claim 23, wherein the AhR agonist is 6-Formylindolo[3,2-b]carbazole (FICZ).

34. The culture media of claim 23, wherein the AhR agonist is Norisoboldine, Pifithrin-α hydrobromide, MeBIO, ITE or 10-C1-BBQ.

35. The culture media of claim 23, wherein the Notch agonist is Yhhu 3792.

36. The culture media of claim 23, wherein the Notch agonist is Jagged 1-2 or DLL1-4.

37. The culture media of claim 23, wherein the HDAC inhibitor is valproic acid (VPA).

38. The culture media of claim 23, wherein the HDAC inhibitor is selected from the group consisting of vorinostat, entinostat, Panobinostat, Trichostatin A, mocetinostat, 4-Phenylbutyric acid, ACY-775, GSK3117391, belinostat, romidepsin, MC1568, tubastatin A, Givinostat, dacinostat, CUDC-101, quisinostat, pracinostat, PCI-34051, droxinostat, abexinostat, RGFP966, AR-42, ricolinostat, tacedinaline, fimepinostat, sodium butyrate, curcumin, M344, tubacin, RG2833, resminostat, divalproex sodium, scriptaid, sodium phenylbutyrate, tubastatin A, sinapinic acid, TMP269, CAY10683, TMP195, UF010, tasquinimod, SKLb-23bb, isoguanosine, NKL22, sulforaphane, BRD73594, citarinostat, suberohydroxamic, BRD3308, splitomicin, HPOB, LMK235, Biphenyl-4-sulfonyl chloride, nexturastat A, BML-210, TC-H106, SR-4370, TH34, Tucidinostat, SIS17, parthenolide, wt161,CAY10603, ACY738, Raddeanin A, Tinostamustine, domatinostat, BG45 and ITSA-1.

39. The culture media of claim 23, which comprises Stem Cell Factor (SCF), thrombopoietin (TPO), Activin A, Vitamin C, lysophosphatidic acid (LPA), 6-Formylindolo[3,2-b]carbazole (FICZ), Yhhu 3792 and valproic acid (VPA).

40. The culture media of claim 39, wherein SCF is present at a concentration of 10 ng/ml, TPO is present at a concentration of 100 ng/ml, Activin A is present at a concentration of 20 ng/ml, Vitamin C is present at a concentration of 100 uM, LPA is present at a concentration of 200 nM, FICZ is present at a concentration of 500 nM, Yhhu 3792 is present at a concentration of 750 nM and VPA is present at a concentration of 150 uM.

41. An isolated cell culture of human CD34+ hematopoietic stem cells (HSCs), the culture comprising: human CD34+ HSCs cultured in a culture media comprising a c-kit ligand, a TPOR agonist, a TGFβ pathway agonist, an antioxidant, a bioactive phospholipid, an aryl hydrocarbon receptor (AhR) agonist, a Notch agonist and a histone deacetylase (HDAC) inhibitor.

42. A method of maintaining human CD34+ long term hematopoietic stem cells (LT-HSCs) comprising culturing human CD34+ HSCs in a culture media comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO).

43. The method of claim 42, wherein the TGFβ pathway agonist is Activin A, the bioactive phospholipid is lysophosphatidic acid (LPA), the AhR agonist is 6-Formylindolo[3,2-b]carbazole (FICZ)and the HDAC inhibitor is valproic acid (VPA).

44. The method of claim 42, wherein the LT-HSCs are CD34⁺ cells that also express CRHBP, HOPX and LMO2.

45. A culture media for maintaining human CD34+ long term hematopoietic stem cells (LT-HSCs) comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO).

46. An isolated cell culture of human CD34+ long term hematopoietic stem cells (LT-HSCs), the culture comprising: human CD34+LT-HSCs cultured in a culture media comprising a TGFβ pathway agonist, a bioactive phospholipid, an aryl hydrocarbon receptor (AhR) agonist and a histone deacetylase (HDAC) inhibitor, wherein the culture media lacks Stem Cell Factor (SCF) and thrombopoietin (TPO).
